(19) United States
(12) Patent Application Publication
(10) Pub. No.: US 2014/0348301 A1
(43) Pub. Date: Nov. 27, 2014

(54) METHOD AND APPARATUS FOR ACQUIRING PLURALITY OF X-RAY FOCUSES

(71) Applicant: SAMSUNG ELECTRONICS CO., LTD., Suwon-si, Gyeonggi-do (KR)

(72) Inventors: Jae-sung Lee, Seongnam-si (KR); Ki-yeo Kim, Suwon-si (KR); Ja-woo Lee, Yongin-si (KR); Min-kook Cho, Hwaseong-si (KR); Byung-sun Choi, Suwon-si (KR)

(21) Appl. No.: 14/368,147

(22) PCT Filed: Dec. 17, 2012
(86) PCT No.: PCT/KR2012/010996

§ 371 Date: Jun. 23, 2014

(30) Foreign Application Priority Data
Dec. 23, 2011 (KR) 10-2011-0141725

(51) Int. Cl. H01J 35/10; A61B 6/00

(52) U.S. Cl. CPC H01J 35/10; A61B 6/4021
USPC 378/125; 378/144

(57) ABSTRACT

A method and apparatus of acquiring a plurality of focal spots for X-rays. The method includes radiating an electron beam from a cathode of a magnetic field generating apparatus toward a movable anode of the magnetic field generating apparatus, and irradiating a target with an X-ray that is produced from the radiated electron beam by the anode. The anode comprises an inclined side in which a part of the inclined side protrudes, and the plurality of focal spots for X-rays are acquired based on the radiated electron beam and the inclined side.

### CROSS-REFERENCE TO RELATED APPLICATIONS

This application is a national stage entry of International Application No. PCT/KR2012/010996 filed on Dec. 17, 2012. This application also claims priority from Korean Patent Application No. 10-2011-0141725, filed on Dec. 23, 2011, in the Korean Intellectual Property Office. The entire disclosures of these applications are incorporated herein by reference.

### BACKGROUND

1. Field

Methods and apparatuses consistent with exemplary embodiments relate to acquiring a plurality of focal spots of a plurality of emitted X-rays, and more particularly, to a method and apparatus for acquiring a plurality of focal spots of a plurality of X-rays produced by an electron beam.

2. Description of the Related Art

Scanning apparatuses that use X-rays are being developed and used as radiographic medical scanning apparatuses. In a scanning apparatus using an X-ray, when an X-ray emitted from an X-ray source passes through a target or object, a scintillator of the scanning apparatus changes the X-ray, which has passed through the target or object, to a visible ray according to the density of the target or object, and the visible ray is changed to an electrical signal via a photodiode of the scanning apparatus. The scanning apparatus using an X-ray represents a digital image of the target through which an X-ray has passed, by using the electrical signal.

When an X-ray beam is radiated to perform a computed tomography (CT) scan, the resolution per pixel of a captured image may be limited to a resolution per pixel corresponding to the size of a cell of an X-ray detector. Accordingly, when several focal spots of X-ray beams are used, an image having a high resolution may be obtained, compared with when a single focal spot is used.

### SUMMARY

A plurality of focal spots for X-rays may be acquired by controlling a rotation speed, for example, of an anode having inclined sides with different heights.

The exemplary embodiments provide a method and an apparatus for acquiring a plurality of focal spots of a plurality of X-rays.

According to an aspect of an exemplary embodiment, there is provided a method of acquiring a plurality of focal spots for X-rays, the method comprising: radiating an electron beam from a cathode of a magnetic field generating apparatus toward a movable anode of the magnetic field generating apparatus; and irradiating a target with an X-ray that is produced from the radiated electron beam by the anode.

The anode may comprise an inclined side in which a part of the inclined side may protrude, and the focal spots for X-rays may be acquired based on the radiated electron beam and the inclined side.

According to another aspect of an exemplary embodiment, there is provided an apparatus for acquiring a plurality of focal spots for X-rays, the apparatus comprising: a cathode which radiates an electron beam toward a movable anode; an anode which produces an X-ray from the radiated electron beam; and an X-ray radiator which radiates the X-ray produced by the anode to a target.

The anode may comprise an inclined side in which a part of the inclined side may protrude, and the focal spots for X-rays may be acquired based on the radiated electron beam and the inclined side.

A movable anode may be rotatable at a variable speed, and an inclined side of the anode may face a direction in which the electron beam is radiated.

A protruding inclined side may comprise a plurality of protruding inclined sides arranged at predetermined intervals.

A height of the protruding inclined side may be greater than a height of a non-inclined side of the anode.

A protruding inclined side may comprise at least two protruding inclined sides that have different heights.

Radiating the electron beam may comprise radiating the electron beam from the cathode to the anode at a fixed angle.

According to an exemplary embodiment, a method of acquiring a plurality of focal spots for X-rays, may further comprise: controlling a rotation speed of the anode; and acquiring at least one focal spot based on the controlled rotation speed and the interval between the plurality of protruding inclined sides.

According to another aspect of an exemplary embodiment, there is provided a computer-readable recording medium having recorded thereon a program for executing the above-described method.

When several focal spots of X-ray beams are used, an image having a high resolution may be obtained, compared to when only a single focal spot is used.

According to an exemplary embodiment, a plurality of focal spots for X-rays may be acquired by controlling a rotation speed, for example, of anode having inclined sides with different heights.

### BRIEF DESCRIPTION OF THE DRAWINGS

The above and/or other aspects will be more apparent by describing certain exemplary embodiments with reference to the accompanying drawings, in which:

FIG. 1 illustrates a resolution per pixel of an image captured by an X-ray having a plurality of focal spots and a resolution per pixel of an image captured by an X-ray having a single focal spot;

FIGS. 2A and 2B are schematic views of a conventional magnetic field generating and controlling apparatus;

FIG. 3 is a flowchart of a method of acquiring a plurality of focal spots of a plurality of X-rays, according to an exemplary embodiment;

FIGS. 4A, 4B, 4C and 4D illustrate an anode according to an exemplary embodiment;

FIGS. 5A, 5B, and 5C illustrates an anode according to another exemplary embodiment;

FIGS. 6A, 6B, 6C, 6D and 6E illustrate an anode including at least two protruding inclined sides that have different heights, according to another exemplary embodiment; and

FIG. 7 is a block diagram of an apparatus for acquiring a plurality of focal spots, according to an exemplary embodiment.

### DETAILED DESCRIPTION OF THE EXEMPLARY EMBODIMENTS

Terminology used herein will now be briefly described, and the exemplary embodiment will be described in detail.

Although general terms being widely used at present were selected as terminology used in the exemplary embodiments while considering the functions of the exemplary embodiments, they may vary according to intentions of one of ordinary skill in the art, judicial precedents, the advent of new technologies, and the like. Terms arbitrarily selected by the applicant may also be used in a specific case. In this case, their meanings can be obtained based on the detailed description of the exemplary embodiments. Hence, the terms must be defined based on the meanings of the terms and the contents of the entire specification, and not by simply stating the terms themselves.

It will be understood that the terms “comprises” and/or “comprising” or “includes” and/or “including” when used in this specification, specify the presence of stated elements, but do not preclude the presence or addition of one or more other elements. Terms such as “ . . . unit” and “module” denote units that process at least one function or operation, and they may be implemented by using hardware, software, or a combination of hardware and software.

In the entire specification, a “captured image” denotes an image of a target that is acquired using X-rays. The target may include an object, a human being, an animal, or a body part thereof. For example, the target may be a chest, an abdomen, arms, legs, or the like of a human or an animal.

In the entire specification, a “user” may be a medical expert, such as a doctor, a nurse, a medical technologist, or a medical image expert, but the exemplary embodiments are not limited thereto.

The exemplary embodiments will now be described more fully with reference to the accompanying drawings. The exemplary embodiments may, however, be embodied in many different forms and should not be construed as being limited to the exemplary embodiments set forth herein. In the drawings, parts irrelevant to the description are omitted for simplicity of explanation, and like numbers refer to like elements throughout.

When an X-ray beam is radiated to perform a computed tomography (CT) scan on a target, the resolution of an image of the scanned target may be limited according to the size of a cell of an X-ray detector. Accordingly, a method of scanning a target by using the focal spots of a plurality of X-ray beams may be used to improve the resolution of a captured image.

For example, a target is scanned using a first X-ray beam produced by emitting a first electron beam in a first direction, to thereby acquire first scan data as a result of the scanning using the first X-ray beam, and the target is scanned using a second X-ray beam produced by emitting a second electron beam in a second direction, to thereby acquire second scan data as a result of the scanning using the second X-ray beam. A captured image of the target may be produced using the first scan data and the second scan data and thus may have an improved resolution compared with an image captured by using an X-ray beam that is radiated with only a single focal spot.

In other words, when several focal spots of an X-ray beam are used, an image having a high resolution may be obtained, as compared to when an X-ray beam having a single focal spot is used.

However, in this method, the focal spot of an X-ray is changed by changing the direction of an electron beam, for example, the angle at which the electron beam is radiated. Thus, a deflector is required to control the direction of the electron beam.

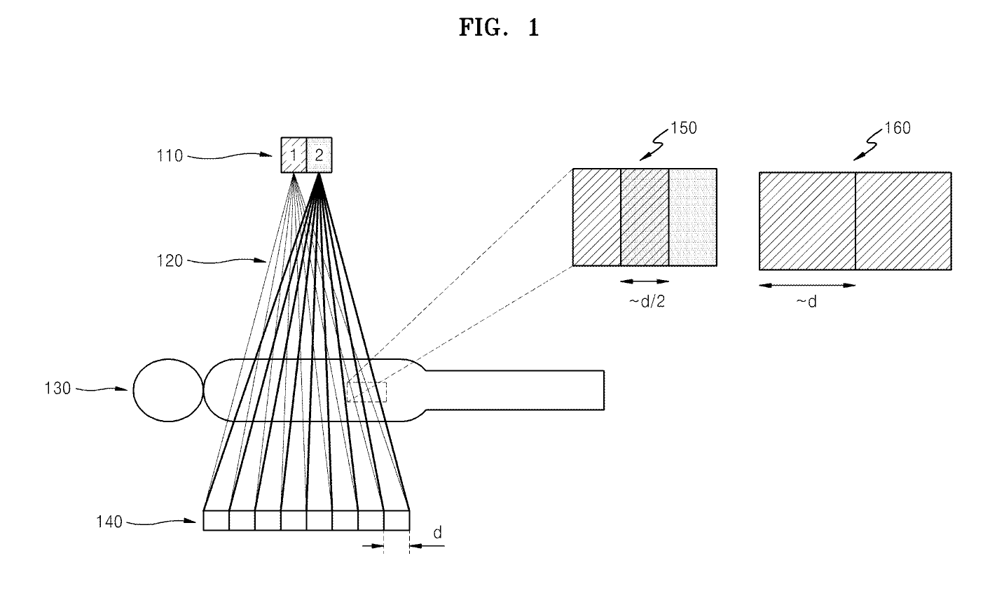

FIG. 1 illustrates a resolution per pixel of an image captured by an X-ray having a plurality of focal spots and a resolution per pixel of an image captured by an X-ray having a single focal spot.

A scanning apparatus using an X-ray generally acquires an image of a target 130 by radiating an X-ray 120 emitted from a source 110 onto the target 130 and detecting the X-ray 120 by using an X-ray detector 140.

If the X-ray 120 emitted from the source 110 has a single focal spot, the size of each pixel from the image of the target 130 may have a value corresponding to the size of each pixel of the X-ray detector 140. For example, when the size of each pixel of the X-ray detector 140 is d, the size of each pixel of the image of the target 130 may also be d as indicated by reference numeral 160.

On the other hand, if the X-ray 120 emitted from the source 110 has a plurality of focal spots, the size of each pixel of the image of the target 130 may be smaller than the size of each pixel of the X-ray detector 140. For example, if the X-ray 120 emitted from the source 110 has two focal spots and the size of each pixel of the X-ray detector 140 is d, the size of each pixel of the image of the target 130 may be d/2 as indicated by reference numeral 150.

In other words, when an X-ray emitted from a source of an X-ray apparatus has a plurality of focal spots, a captured image having a higher resolution may be obtained, as compared to when the X-ray emitted from the source of the X-ray apparatus has a single focal spot.

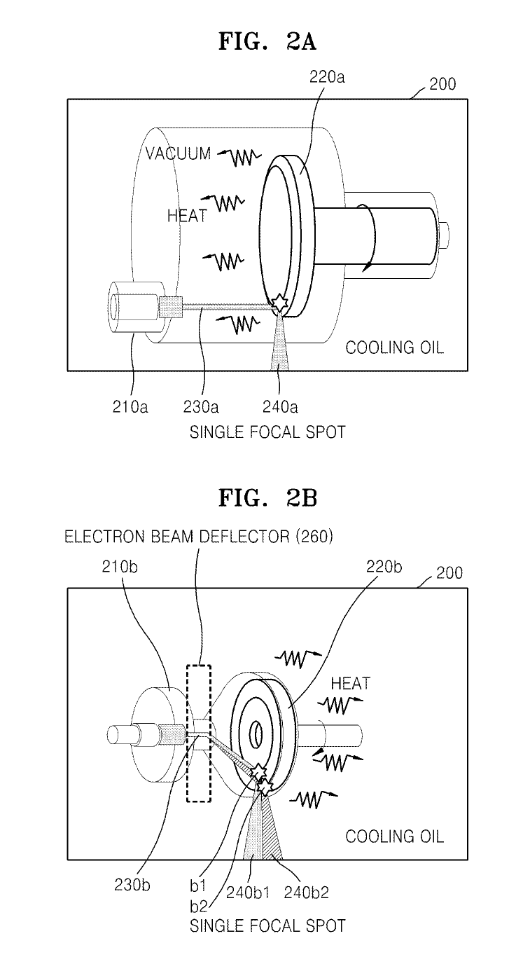

FIGS. 2A and 2B are schematic views of a conventional magnetic field generating and controlling apparatus.

FIG. 2A illustrates a process in which a magnetic field generating apparatus generates an X-ray, and FIG. 2B illustrates a process in which a magnetic field generating apparatus, including an electron beam deflector, generates an X-ray.

General generation of an X-ray by a magnetic field generating and controlling apparatus 200 is as follows:

An electron beam 230a may be generated between a cathode 210a and an anode 220a of a magnetic field generating apparatus 200, and an electron beam 230b may be generated between a cathode 210b and an anode 220b of the magnetic field generating apparatus 200. The electron beam 230a emitted from the cathode 210a may produce an X-ray beam 240a by hitting the anode 220a, and the electron beam 230b emitted from the cathode 210b may produce X-ray beams 240b1 and 240b2 by hitting the anode 220b.

In general, the anodes 220a and 220b may be constructed so as to rotate. Heat is generated when the electron beams 230a and 230b, respectively emitted from the cathodes 210a and 210b, hit the anodes 220a and 220b, respectively. To prevent the magnetic field generating apparatus 200 from being damaged by the generated heat, the cathodes 210a and 210b and the anodes 220a and 220b may be cooled off by using cooling oil.

Referring to FIG. 2B, which illustrates the magnetic field generating apparatus including the electron beam deflector 260, the electron beam 230b generated by the cathode 210b may be radiated onto the anode 220b at various angles due to the use of the electron beam deflector 260.

In other words, the electron beam 230b may hit various spots on the anode 220b. The X-ray beams 240b1 and 240b2 may have a plurality of focal spots. For example, the electron beam 230b may hit a spot b1 on the anode 220b via the electron beam deflector 260 to produce the X-ray 240b1. The electron beam 230b may hit a spot b2 on the anode 220b via the electron beam deflector 260 to produce the X-ray 240b2.

As such, an X-ray having a plurality of focal spots may be acquired using an electron beam deflector 260, and thus the magnetic field generating apparatus of FIG. 2B may be used.

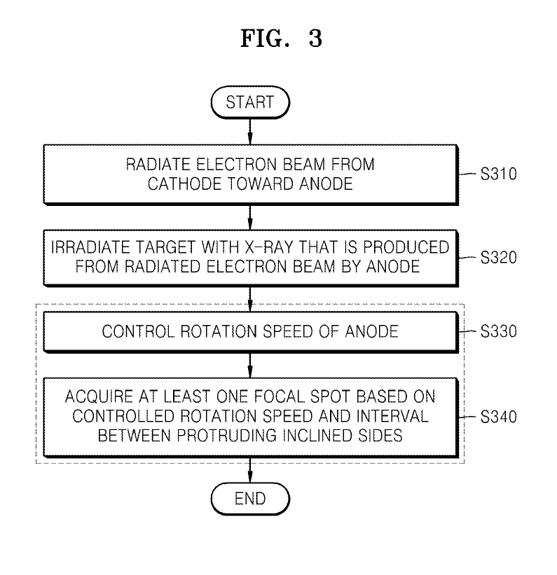

FIG. 3 is a flowchart of a method of acquiring focal spots of a plurality of X-rays, according to an exemplary embodiment.

Referring to FIG. 3, the method may include operation S310 of radiating an electron beam from a cathode of a magnetic field generating apparatus toward a movable anode of the magnetic field generating apparatus, and operation S320 of irradiating a target with an X-ray that is produced from the radiated electron beam by the anode.

The anode may include an inclined side, and a part of the inclined side may protrude.

The anode is movable and may rotate at a variable speed, and the inclined side of the anode may face the direction in which the electron beam is radiated.

The protruding inclined side of the anode may include a plurality of inclined sides that protrude at predetermined intervals.

The height of the protruding inclined side may be greater than the height of a non-protruding inclined side of the anode.

The protruding inclined side may include at least two protruding inclined sides that have different heights.

Operation S310 of radiating the electron beam from the cathode may include radiating the electron beam from the cathode to the anode at a fixed angle.

The method may further include operation S330 of controlling the rotation speed of the anode and operation S340 of acquiring at least one focal spot based on the controlled rotation speed and the interval between the protruding inclined sides.

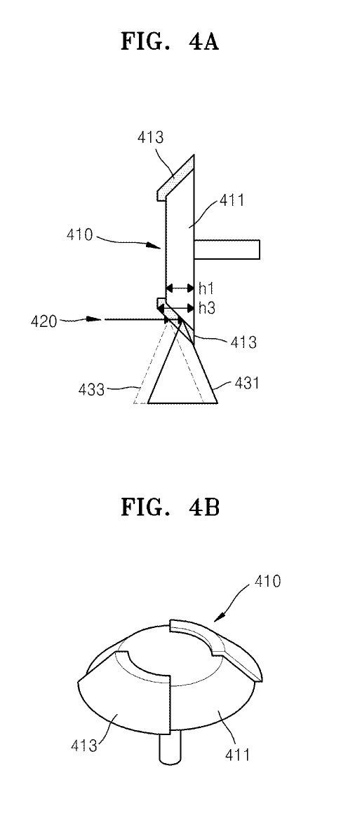

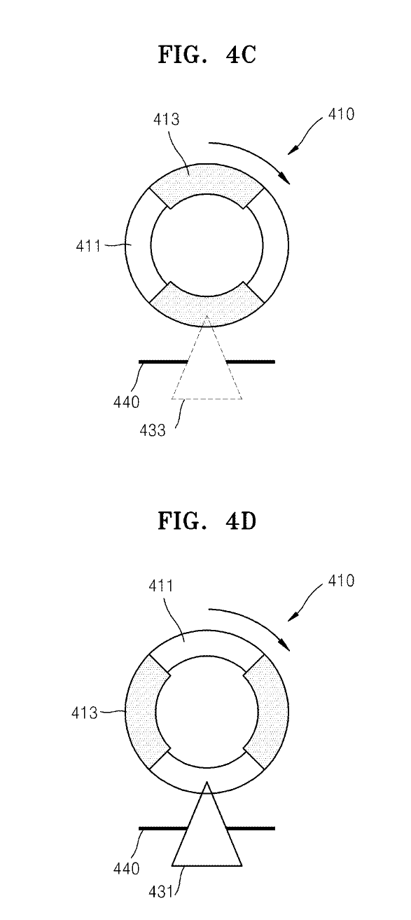

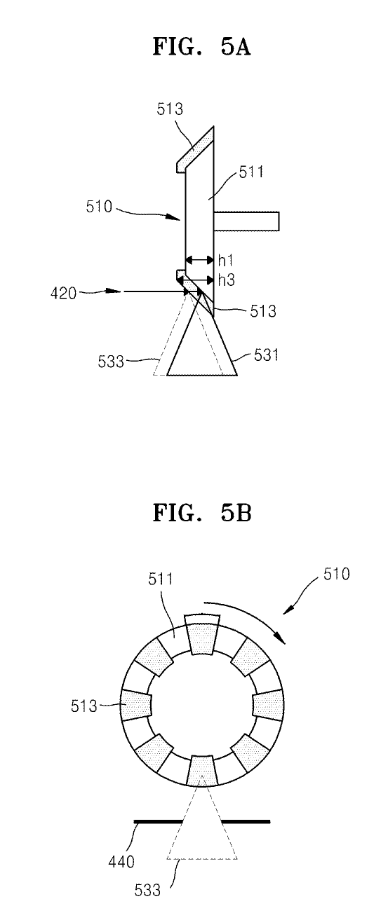

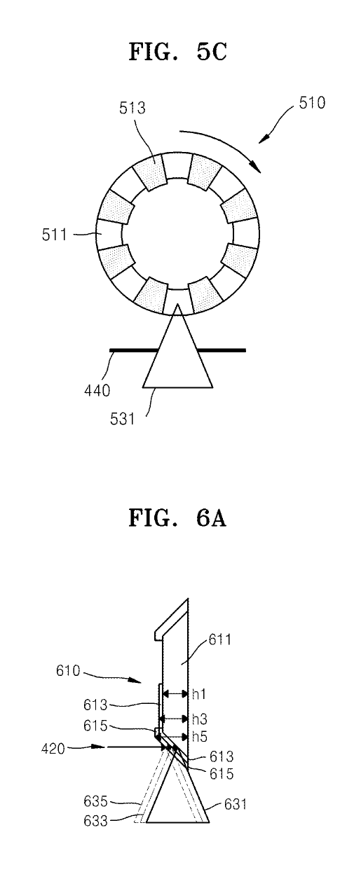

The method will now be described in greater detail with reference to FIGS. 4A-4D and 5A-5C.

FIGS. 4A-4D illustrate an anode 410 according to an exemplary embodiment. FIGS. 5A-5C illustrate an anode 510 according to another exemplary embodiment.

Referring to FIG. 4B, the anode 410 may include inclined sides 411 and 413, which are inclined to face the direction in which an electron beam 420 is radiated. The anode 410 may include the protruding inclined side 413 on the edge of the anode 410.

According to an exemplary embodiment, the protruding inclined side 413 may include a plurality of protruding inclined sides arranged at predetermined intervals. A height h3 of the protruding inclined side 413 may be greater than a height h1 of the non-protruding inclined side 411.

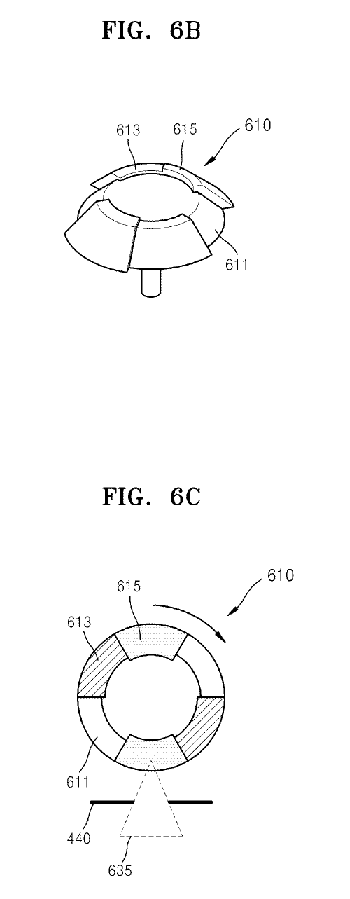

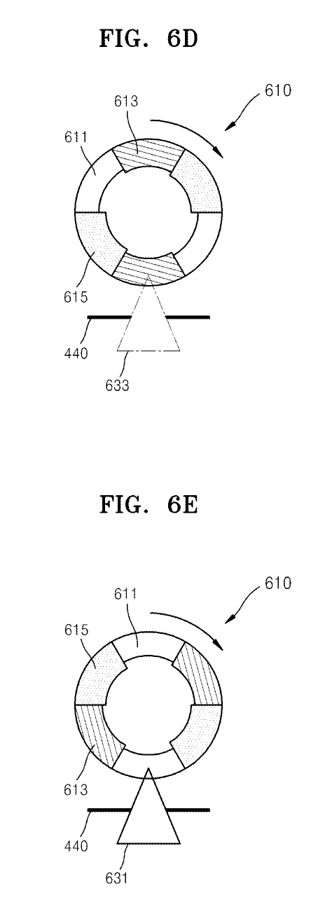

The protruding inclined side 413 of the anode 410 may include at least two protruding inclined sides that have different heights. For example, the anode 410 may include an inclined side having a first height h1, a protruding inclined side having a second height h3, and a protruding inclined side having a third height h5 (see FIGS. 6A-6E). The heights h1, h3, and h5 may satisfy an inequality of h1<h3<h5.

According to an exemplary embodiment, the anode 410 is movable and may rotate at a variable speed. For example, as the anode 410 rotates, the electron beam 420 may hit different spots on the anode 410 according to different heights of the inclined sides.

For example, referring to FIG. 4C, when the electron beam 420 hits the protruding inclined side 413 of the anode 410, an X-ray 433 may be produced by the electron beam 420 and the anode 410. In other words, the electron beam 420 may produce the X-ray 433 by hitting the anode 410, and the generated X-ray 433 may be radiated onto a target via a collimator 440.

Referring to FIG. 4D, when the electron beam 420 hits the non-protruding inclined side 411 of the anode 410, an X-ray 431 may be produced by the electron beam 420 and the anode 411, and the generated X-ray 431 may be radiated onto the target via the collimator 440.

However, in this case, as illustrated in FIG. 4A, the electron beam 420 proceeds further in the radiation direction of the electron beam 420 to hit the non-protruding inclined side 411 than to hit the protruding inclined side 413 of the anode 410. Thus, the X-ray 431 located behind the X-ray 433, which is generated by the protruding inclined side 413, in the radiation direction of the electron beam 420 may be generated.

In other words, when an electron beam is radiated while rotating the anode 410 having inclined sides with different heights, a plurality of X-rays having different focal spots may be acquired according to the inclined sides having different heights.

As described above, the anode 410 is movable and may rotate at a variable speed. For example, a sampling rate for a general Computed Tomography (CT) scan is 3000 samples per second, as required to construct at least one frame.

According to an exemplary embodiment, for example, if an anode 410 having two protruding inclined sides 413 is used, four samples are acquired during each rotation due to the alternating hits of the electron beam 420 on the protruding inclined sides 413 and the non-protruding inclined side 411 of the anode 410. Accordingly, the rotation rate of the anode 410 may be about 45,000 rpm.

In view of the fact that the rotation rate of a general anode is about 10,000 rpm, the anode 510 according to another exemplary embodiment of FIGS. 5A-5C may be considered.

According to another exemplary embodiment, the number of protruding inclined sides 513 included in the anode 510 may be controlled in consideration of the size and rotation speed of the anode 510. Even when the number of protruding inclined sides 513 included in the anode 510 varies, a plurality of focal spots may be acquired because the inclined sides 513 and 511 of the anode 510 have different heights, in the same way as described in FIGS. 4A-4D regarding acquiring a plurality of focal spots.

When a sampling rate for a general CT scan is 3000 samples per second as described above, if eight protruding inclined sides 513 are used as shown in FIGS. 5A-5C, 16 samples may be acquired during each rotation of the anode 510. Accordingly, the rotation rate of the anode 510 may be about 11,250 rpm. Since the rotation rate of the anode 510 is approximately 10,000 rpm, which is the rotation rate of a general anode, the anode 510 is highly compatible with conventional apparatuses.

According to an exemplary embodiment, a plurality of protruding inclined sides may be arranged on an anode at predetermined intervals. For example, the general size of an anode has a diameter of about 20 centimeters. Accordingly, the width of each protruding inclined side of the anode may be obtained by dividing the circumference of the anode by the number of protruding inclined sides. For example, when an anode includes eight protruding inclined sides, the width of each protruding inclined side may be about 3.9 centimeters.

According to an exemplary embodiment, in some cases, an X-ray having a single focal spot may be radiated via adjustment of the rotation rate of an anode. For example, when eight protruding inclined sides 513 are used as illustrated in FIGS. 5A-5C, the rotation rate of the anode 510 may be controlled so that an electron beam is radiated only to either the protruding inclined sides 513 or the non-protruding inclined side 511 of the anode 510, thereby producing an X-ray 533 or 531 having a single focal spot.

FIGS. 6A-6E illustrate an anode 610 including at least two protruding inclined sides that have different heights, according to another exemplary embodiment.

The anode 610 may have a pattern of, for example, three inclined sides having different heights. The pattern may indicate a design in which an arrangement of inclined sides is repeated at predetermined intervals on the edge of an anode, as illustrated in FIGS. 6A-6E.

Since the anode 610 includes inclined sides 613 and 615 having three different heights, X-rays 631, 633, and 635 having three different focal spots may be produced. The production of the X-rays 631, 633, and 635 having different focal spots is the same as described above with reference to FIGS. 4A-4D and 5A-5C. Specifically, the X-rays 631, 633, and 635 have different focal spots depending on which side 611, 613 and 615 of the anode the electron beam 420 hits. The anode 610 may include an inclined side having a first height h1, a protruding inclined side having a second height h3, and a protruding inclined side having a third height h5. The heights h1, h3, and h5 may satisfy an inequality of h1<h3<h5.

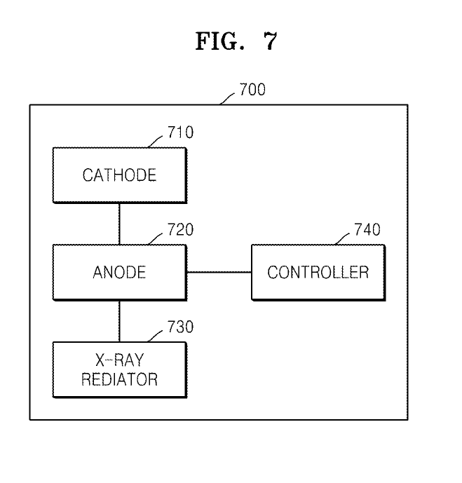

FIG. 7 is a block diagram of an apparatus 700 for acquiring a plurality of focal spots, according to an exemplary embodiment.

Referring to FIG. 7, the apparatus 700 may include a cathode 710, which radiates an electron beam toward an anode 720 that is movable, the anode 720, which produces an X-ray due to the radiated electron beam, and an X-ray radiator 730, which radiates the X-ray produced by the anode 720 to a target.

The anode 720 may include an inclined side, and a part of the inclined side may protrude. A plurality of X-ray focal spots for X-rays may be acquired based on the radiated electron beam and the inclined side of the anode 720.

The anode 720 is movable and may rotate at a variable speed, and the inclined side of the anode 720 may face the direction in which the electron beam is radiated.

A protruding inclined side of the anode 720 may include a plurality of protruding inclined sides arranged at predetermined intervals.

The height of the protruding inclined side of the anode 720 may be greater than that of a non-protruding inclined side of the anode 720.

The protruding inclined side of the anode 720 may include at least two protruding inclined sides that have different heights.

The cathode 710 may radiate the electron beam toward the anode 720 at a fixed angle.

The apparatus 700 may further include a controller 740, which controls the rotation speed of the anode 720. At least one focal spot may be acquired based on the rotation speed of the anode 720 controlled by the controller 740 and the interval between the protruding inclined sides on the anode 720.

The above-described exemplary embodiments may be written as computer programs and may be implemented in general-use digital computers that execute the programs using a computer-readable recording medium.

Examples of the computer-readable recording medium include magnetic storage media (e.g., ROM, floppy disks, hard disks, etc.), optical recording media (e.g., CD-ROMs or DVDs).

While the exemplary embodiments have been particularly shown and described, it will be understood by those of ordinary skill in the art that various changes in form and details may be made therein without departing from the spirit and scope as defined by the appended claims. Therefore, the exemplary embodiments should be considered in descriptive sense only and not for purposes of limitation. Therefore, the scope of the exemplary embodiments is defined not by the detailed description of exemplary embodiments, but by the appended claims, and all differences within the scope will be construed as being included in the exemplary embodiments.

1. A method of acquiring a plurality of focal spots for X-rays, the method comprising:
radiating an electron beam from a cathode of a magnetic field generating apparatus toward a movable anode of the magnetic field generating apparatus; and
irradiating a target with an X-ray that is produced from the radiated electron beam by the anode,
wherein the anode comprises an inclined side in which a part of the inclined side protrudes, and the plurality of focal spots for X-rays are acquired based on the radiated electron beam and the inclined side.

2. The method of claim 1, wherein
the movable anode is rotatable at a variable speed, and
wherein the inclined side of the anode is configured to face a direction in which the electron beam is radiated.

3. The method of claim 2, wherein the protruding inclined side comprises a plurality of protruding inclined sides arranged at predetermined intervals.

4. The method of claim 3, wherein a part of the inclined side is a non-protruding side; and
wherein a height of the protruding inclined side is greater than a height of the non-protruding inclined side of the anode.

5. The method of claim 4, wherein the protruding inclined side comprises at least two protruding inclined sides that have different heights.

6. The method of claim 1, wherein the radiating of the electron beam comprises radiating the electron beam from the cathode to the anode at a fixed angle.

7. The method of claim 3, further comprising:
controlling a rotation speed of the anode; and
acquiring at least one focal spot based on the controlled rotation speed and an interval between the plurality of protruding inclined sides.

8. An apparatus for acquiring a plurality of focal spots for X-rays, the apparatus comprising:
a cathode configured to radiate an electron beam toward a movable anode;
an anode configured to produce an X-ray from the radiated electron beam; and
an X-ray radiator configured to radiate the X-ray produced by the anode to a target,
wherein the anode comprises an inclined side in which a part of the inclined side protrudes, and the plurality of focal spots for X-rays are acquired based on the radiated electron beam and the inclined side.

9. The apparatus of claim 8, wherein
the movable anode is configured to rotate at a variable speed, and
wherein the inclined side of the anode is configured to face a direction in which the electron beam is radiated.

10. The apparatus of claim 9, wherein the protruding inclined side comprises a plurality of protruding inclined sides arranged at predetermined intervals.

11. The apparatus of claim 10,
wherein a part of the inclined side is a non-protruding side; and
wherein a height of the protruding inclined side is greater than a height of the non-protruding inclined side of the anode.

12. The apparatus of claim 11, wherein the protruding inclined side comprises at least two protruding inclined sides that have different heights.

13. The apparatus of claim 8, wherein the cathode radiates the electron beam toward the anode at a fixed angle.

14. The apparatus of claim 10, further comprising a controller configured to control a rotation speed of the anode,
wherein at least one focal spot is acquired based on the rotation speed controlled by the controller and the interval between the plurality of protruding inclined sides.

15. A computer-readable recording medium having recorded thereon a program for executing a method of acquiring a plurality of focal spots for X-rays, the method comprising:
radiating an electron beam from a cathode of a magnetic field generating apparatus toward a movable anode of the magnetic field generating apparatus; and
irradiating a target with an X-ray that is produced from the radiated electron beam by the anode,
wherein the anode comprises an inclined side in which a part of the inclined side protrudes, and the plurality of focal spots for X-rays are acquired based on the radiated electron beam and the inclined side.

16. A magnetic field generating apparatus comprising:
a movable anode comprising a plurality of sides having varying height;
wherein the anode comprises an inclined side in which a part of the inclined side protrudes, and a plurality of focal spots for X-rays are acquired based on a radiated electron beam which hits the inclined side.

17. A method of acquiring a plurality of focal spots for X-rays, the method comprising:
radiating an electron beam from a cathode of a magnetic field generating apparatus toward a movable anode of the magnetic field generating apparatus; and
irradiating a target with an X-ray that is produced from the radiated electron beam from the anode,
wherein the anode comprises an inclined side in which a part of the inclined side protrudes, and the plurality of focal spots for X-rays are acquired based on the radiated electron beam which hits the inclined side.

18. An anode for a magnetic field generating apparatus, the anode comprising:
a plurality of non-protruding inclined sides; and
a plurality of protruding inclined sides;
wherein the plurality of non-protruding inclined sides and the plurality of protruding inclined sides vary in height;
wherein a plurality of focal spots for X-rays are acquired based on a radiated electron beam which hits the plurality of non-protruding inclined sides and the plurality of protruding inclined sides during rotation of the anode.
